(19) United States
(12) Patent Application Publication
(10) Pub. No.: US 2015/0030661 A1
(43) Pub. Date: Jan. 29, 2015

(54) Multilayer Films and Uses Thereof

(71) Applicant: Massachusetts Institute of Technology, Cambridge, MA (US)

(72) Inventors: Katharina Ribbeck, Cambridge, MA (US); Thomas Crouzier, Somerville, MA (US)

(21) Appl. No.: 14/376,714

(22) PCT Filed: Feb. 6, 2013
(86) PCT No.: PCT/US2013/024978

§ 371 Date: Aug. 5, 2014

Related U.S. Application Data
(60) Provisional application No. 61/595,593, filed on Feb. 6, 2012.

(51) Int. Cl. A01N 25/34; A61K 9/70; B01D 15/32; A61K 47/42; C12N 11/10; G01N 33/566; A01N 43/16

(52) U.S. Cl. CPC A01N 25/34; G01N 33/566; A61K 9/70; A01N 43/16; A61K 47/42; C12N 11/10; B01D 15/32; G01N 2333/36
USPC 424/443; 428/534; 436/501; 435/7.2; 435/7.31; 435/7.32; 530/413; 427/414

(57) ABSTRACT

The invention provides, inter alia, multilayer films of alternating layers of a glycosylated polymer (e.g. a mucin) and a lectin, as well as methods of making and using these films. The films can be adapted for, inter alia, delivery of a biologically active agent providing a non-toxic substrate to support cell growth, replication, and/or maintenance as well as detecting a microorganism and/or reducing microorganism adherence to a surface.

### RELATED APPLICATION

This application claims the benefit of U.S. Provisional Application No. 61/595,593, filed on Feb. 6, 2012.

The entire teachings of the above application are incorporated herein by reference.

### BACKGROUND

Biologically compatible surfaces offer a wide variety of applications to functionalize surfaces, from drug release to supporting (or inhibiting) cell attachment and/or growth. Such surfaces however can be complicated, difficult, and costly to make and may not be durable (e.g., they may disassemble at extreme pH or ionic strength). Accordingly, a need exists for durable, cost-effective, biologically compatible surfaces that are easy to engineer.

### SUMMARY OF THE INVENTION

The invention provides, inter alia, durable, cost-effective, biologically compatible surfaces that are easy to engineer.

In one aspect, the invention provides multilayer films comprising alternating layers of a glycosylated polymer and a lectin where the lectin crosslinks the glycosylated polymers. In some embodiments, the glycosylated polymer is mucin, chondriotin sulfate, glycogenin in combination with concanavalin A, or a combination thereof. In more particular embodiments, the mucin is porcine gastric mucin, bovine submaxillary mucin (BSM) or a combination thereof. In still more particular embodiments, the porcine gastric mucin is purified porcine gastric mucin composed primarily of MUC5AC, MUC2, MUC5B, and MUC6. In some embodiments, the lectin is wheat germ agglutinin (WGA), jacalin or a combination thereof.

In some embodiments, the multilayer films provided by the invention are able to bind a microorganism. “Microorganism” encompasses bacteria, viruses, fungi, and protists. In particular embodiments, the microorganism bound has a receptor or other cell-surface molecule that binds a mucin or a lectin. For example, the microorganism may have a cell-surface molecule that comprises a saccharide epitope bound by a lectin.

In certain embodiments, the multilayer films provided by the invention formed on a polystyrene or glass substrate. In still more particular embodiments, the film is lectin-depleted.

In certain embodiments, the multilayer films provided by the invention further comprising one or more additional agents attached to one or more layers of the multilayer film. In some particular embodiments, the one or more additional agents is a positively charged molecule that is bound to one or more layers of the glycosylated polymer. In still more particular embodiments, the positively charged molecule is a polycationic polymer, a growth factor, an antimicrobial peptide, a virus, a drug or a combination thereof. In other embodiments, the one or more additional agents are one or more labels that is attached to one or more layers of the lectin. In more particular embodiments, the one or more labels is biotin or avidin.

In some embodiments, the multilayer films provided by the invention further comprise a substrate onto which the multilayer film is deposited. In more particular embodiments, the substrate is a polystyrene surface, a gold covered quartz crystal or a polystyrene covered crystal.

In certain embodiments, the multilayer films provided by the invention are biocompatible. In some embodiments, the film is insensitive to ionic strength conditions. In more particular embodiments, the ionic strength conditions comprises about 5M NaCl, or an equivalent ionic strength. In certain embodiments, the film is insensitive to extreme pH conditions. In some embodiments, the films provided by the invention tolerate a pH of about 3, 4, 5, 6, 7, 8, 9, 10 or 11. In more particular embodiments, the films are resistant to both high ionic strength and extreme pH.

In certain embodiments, the multilayer films provided by the invention include about 2, 3, 4, 5, 6, 7, 8, 9, 10, 11, 12, 13, 14, 15, 16, 17, 18, 19, 20 or more bilayers of alternating layers of the polymer and the lectin. In some particular embodiments, the final layer (i.e. the external, exposed layer) is a lectin layer. In other particular embodiments, the final layer is a glycosylated polymer layer.

In some embodiments, the film is lyophilized or otherwise dried, e.g., by air drying. In other embodiments, the film is hydrated.

In some embodiments, in the films provided by the invention, the lectin is conjugated to an additional biologically active agent. In more particular embodiments, the lectin and additional biologically active agent are covalently conjugated. In other particular embodiments, the lectin and additional biologically active agent are non-covalently conjugated.

In certain embodiments, the multilayer film provided by the invention further comprises a ligand of the lectin, preferably wherein the lectin has a higher affinity for the ligand than the lectin's affinity for the glycosylated polymer. The multilayer film provided by the invention treated in this way result in lectin-depleted films, which are encompassed by the invention as well.

In some embodiments, the multilayer film of any one of the preceding claims further comprises materials to support the maintenance, growth, or replication of a cell in culture (e.g. as a synthetic extracellular matrix).

In certain embodiments, the multilayer film provided by the invention may comprise animal cells reversibly bound to the outer surface of the film. In more particular embodiments, the animal cell is releasable by contacting the film with a saccharide ligand of the lectin.

In another aspect, the invention provides pharmaceutical compositions comprising any of the multilayer films provided by the invention.

In another aspect, the invention provides methods of producing a multilayer film comprising alternately depositing a glycosylated polymer with depositing a lectin on a substrate. In some embodiments, the methods further comprise one or more steps of washing the film after depositing of each layer onto the substrate.

In certain embodiments of these methods, the polymer is a mucin, chondriotin sulfate, glycogen in combination with ConA or a combination thereof. In more particular embodiments, the mucin is porcine gastric mucin, bovine submaxillary mucin (BSM) or a combination thereof. In other particular embodiments, the polymer is a mucin, which is applied at a concentration of about 0.1 mg/mL to about 2.0 mg/mL, e.g. about 0.2 to about 1.0 mg/mL.

In some embodiments, the lectin is wheat germ agglutinin (WGA), jacalin or a combination thereof. In certain embodiments of the methods provided by the invention, the lectin is applied at a concentration of about 0.05 to about 2.0 mg/mL; e.g. about 0.01 to about 1.0 mg/mL.

In certain embodiments of the methods provided by the invention, the final layer is a lectin layer. In other particular embodiments, the final layer is a polymer layer.

In some embodiments, the methods provided by the invention may further comprise contacting the multilayer film with a ligand of the lectin and maintaining the multilayer film under conditions in which all or a portion of the lectin in a final layer or layers of the multilayer film is released, thereby exposing charged groups of an underlying polymer layer. In more particular embodiments, the polymer is porcine gastric mucin, the lectin is wheat germ agglutinin and the ligand is N-Acetyl-D-Glucosamine; or the polymer is bovine submaxillary mucin (BSM), the lectin is jacalin and the ligand is melibiose.

In certain embodiments, the methods provided by the invention further comprise contacting the multilayer film with one or more additional agents. In more particular embodiments, one or more of the additional agents is a positively charged molecule that is bound to one or more layers of the glycosylated polymer. In still more particular embodiments, the positively charged molecule is a polycationic polymer, a growth factor, an antimicrobial peptide, a drug or a combination thereof. In certain embodiments, the methods provided by the invention further comprise the step of maintaining the multilayer film under conditions in which the one or more agents are released from the multilayer film. In particular embodiments the conditions comprise altering ionic conditions, pH conditions or a combination thereof. In other particular embodiments, the methods further include the steps of contacting the multilayer film with one or more additional agents after the one or more agents are released from the multilayer film and maintaining the multilayer film under conditions in which the one or more additional agents binds to the multilayer film. In certain embodiments, the one or more additional agents are one or more labels that is attached to one or more layers of the lectin. In more particular embodiments, the one or more labels is biotin or avidin. In still more particular embodiments, the label is biotin. In yet more particular embodiments, the methods provided by the invention include the step where the film is contacted with a composition comprising one or more molecules of streptavidin attached to one or more molecules of interest, thereby producing a combination, and maintaining the combination under conditions in which the streptavidin that is attached to the molecule of interest binds to the biotin attached to the one or more layers of the lectin.

In some embodiments of the methods provided by the invention, the substrate is a polystyrene surface, a gold covered quartz crystal or a polystyrene covered crystal.

In certain embodiments of the methods provided by the invention, the multilayer film is biocompatible.

In some embodiments, the film used in the methods provided by the invention The method of any one of claims 31-53 wherein the multilayer film comprises 2, 3, 4, 5, 6, 7, 8, 9, 10, 11, 12, 13, 14, 15, 16, 17, 18, 19, 20 or more bilayers of alternating layers of the polymer and the lectin.

In some embodiments the film used in the methods provided by the invention is insensitive to ionic strength conditions, such as ionic strength conditions are equivalent to about 5M NaCl. In other embodiments, the film is insensitive to extreme pH conditions. In some embodiments the pH is about 3, 4, 5, 6, 7, 8, 9, 10 or 11. In more particular embodiments, the films are resistant to both high ionic strength and extreme pH.

In another aspect, the invention provides methods of detecting or isolating a molecule of interest by attaching streptavidin to the molecule of interest, contacting the molecule of interest with a multilayer film provided by the invention, producing a combination; and maintaining the combination under conditions in which the biotin of the multilayer film binds to the streptavidin of the molecule of interest, thereby detecting or isolating a molecule of interest.

In another aspect, the invention provides a method of delivering an agent to an individual in need thereof comprising introducing a multilayer film provided by the invention that includes an additional agent to the individual, thereby delivering the agent.

In yet another aspect, the invention provides a multilayer film produced by any one of the methods provided by the invention.

In yet another aspect, the invention provides a method of reducing bacterial adhesion to a surface, including applying the multilayer substrates provided by the invention to the surface and depleting the lectin from the substrate. In more particular embodiments, the surface comprises glass, a plastic (such as polystyrene), or a combination thereof.

In another aspect, the invention provides any of the multilayer films provided by the invention with a bound microorganism (e.g., a bacteria, virus, fungus, or protist, preferably wherein the microorganism has a receptor or other cell-surface molecule that binds a mucin). Preferably the films for these embodiments are not lectin-depleted. In a related aspect, the invention provides methods of binding and, optionally, detecting, a microorganism comprising contacting the microorganism with the foregoing multilayer films. In particular embodiments, the multilayer films used in these methods are not lectin depleted. Therefore, in a related aspect the invention also provides a biosensor comprising the multilayer films described herein. Optionally the biosensor may be contained within a kit, optionally including one or more of suitable positive control, suitable negative control, and instructions for use. Therefore, in yet another related aspect, the invention provides methods of detecting a microorganism comprising contacting a sample suspected of containing one or more microorganisms with the biosensors provided by the invention to form a complex between the microorganism and the biosensor and detecting the complex, thereby detecting the microorganism.

### BRIEF DESCRIPTION OF THE DRAWINGS

FIGS. 1A-1D. Multilayer film components. Mucin is schematically represented by a protein core with regions that are densely glycosylated. (1A). The polysaccharides attached by O-linkages are mostly composed of N-Acetyl-D-Galactosamine (1B), N-Acetyl-D-Glucosamine (1C) and N-acetylneuraminic acid (1 D).

FIGS. 2A-2B. Multilayer film growth. Hydrated thickness was obtained by QCM-D measurements, as a function of layer number for (Mucin/WGA (wheat germ agglutinin)) multilayer films built with 0, 200 or 400 mM NaCl (2A). As a comparison, the same measurement was performed for the electrostatic-based (Mucin/PLL) multilayer. The growth was limited to 8 bilayers due to repeated premature loss of the QCM-D signal (2B).

FIGS. 3A-3D. Multilayer film morphology and hydration. (Mucin-Alexa488/WGA)12 multilayers observed with 10× epifluorescence microscopy (A), 100× epifluorecence microscopy (B) and AFM (C). The hydration of the (Mucin/WGA)₁₂ multilayers were measured for multilayers built in buffer with either 0, 200 or 400 mM NaCl (D).

FIG. 4. Multilayer film resistance to degradation. (Mucin-Alexa488/WGA)₁₂ and (Mucin/WGA-Alexa488)12 multilayers were tested for their resistance to extreme pH and salt concentrations. The reported values are the percentage of remaining multilayer components after the treatment. Both WGA and mucins resisted well to these treatments, except in the presence of pH 12.

FIGS. 5A-5C. GlcNAc induced WGA release. (Mucin-Alexa488/WGA)₁₂ and (Mucin/WGA-Alexa488)₁₂ were assembled. The composition of the multilayer is described as the mass ratio between mucin and WGA in each individual buildup step. After buildup, 3 consecutive GlcNAc treatments were applied to the multilayer, with intermediate washing steps. This is a representative experiment of two independent experiments, error bars are omitted for the sake of clarity. The full data, with standard deviation bars is presented in FIG. 10 (5A). The percentage of remaining mucin and WGA after WGA-mediated release is plotted. Error bars represent standard deviations from three independent experiments (5B). Schematic representation of the GlcNAc-induced release of WGA from the multilayers, leaving only a fraction of strongly-bound WGA to maintain the multilayer's integrity (5C).

FIGS. 6A-6B. Multilayer film loading with PLL. The PLL incorporated in (Mucin/WAG)₁₂ multilayers finished with a layer of either mucin or WGA was quantified and is indicated as the mass ratio of PLL to mucin in the multilayer. The release of WGA induced by GlcNAc significantly increased this ratio (6A). The PLL loading in a GlcNAc treated multilayer could be reversed by 5 M NaCl treatments, then reloaded with PLL over several cycles (6B).

FIG. 7. Multilayer film cytotoxicity. Viability of HeLa cells after 24 hours of culture on (Mucin/WGA)₁₂ multilayers with either mucin or WGA as a final layer in contact with the cells. Both multilayer films were treated or not with GlcNAc to release WGA were tested.

FIGS. 8A-8B: Mucin coatings cannot spontaneously auto-assemble into multilayer. QCM-D measurement of mucin adsorption (0.2 mg/mL, in 0 mM NaCl 20 mM Hepes, pH 7.4), followed by a washing step (0 mM NaCl, 20 mM Hepes) and a second mucin adsorption in similar conditions (arrow). The raw frequency change is shown (8A), as well as the modeled thickness (8B). No significant change in frequency nor modeled thickness could be observed after introducing mucin over a mucin coating.

FIG. 9: (Mucin/WGA)₁₂ films were built either on gold coated QCM-D crystals (Qsense, Sweden) or polystyrene coated crystals (Q-sense, Sweden). The two growth curves were very similar, suggesting that differences in surface properties have insignificant effect on film growth.

FIG. 10: The composition of the multilayer (Mucin is described as the mass ratio between mucin and WGA in each individual buildup step. After buildup, 3 consecutive GlcNAc treatments were applied to the multilayer, with intermediate washing steps. This is the same data is presented in FIG. 5, however here, the average of two independent experiments and standard deviation is plotted.

FIGS. 11A-11B: Graphs of layer number vs. fluorescence of chondroitin sulfate/WGA and BSM/jacalin. The WGA and jacalin lectins were labeled with fluorescein.

FIG. 12: A bar graph showing the percentage of remaining polymer at 5M NaCl.

FIG. 13: A schematic showing the use of the multilayer films provided herein to capture or detect a molecule of interest.

FIG. 14: A graph showing the amounts of biotin-fitc incorporated into 1, 2, and 11 layers of a jacalin-biotin/avidin/biotin-fitc sequence.

FIG. 15: A graph of percentages of BSM, Jacalin, and Biotin-FITC released with 30 minute incubations of 150 ul melibiose.

FIG. 16: A graph of uM melibiose vs. % of biotin-fitc release.

FIG. 17 illustrates the experimental protocol used in Example 3.

FIGS. 18A and 18B are bar graphs that illustrate the adhesion of S. aureus (18A) and E. coli (18B), before lectin release. “PGM” means the multilayer films are ended by pig gastric mucin. “WGA” means that the films are ended by wheat germ agglutinin.

FIGS. 19A and 19B are bar graphs that illustrate the adhesion of E. coli (19A) and S. aureus (19B), after lectin release. “PGM” means the multilayer films are ended by pig gastric mucin. “WGA” means that the films are ended by wheat germ agglutinin.

### DETAILED DESCRIPTION OF THE INVENTION

A description of example embodiments of the invention follows.

Multilayer films of biopolymers are attractive methods to exploit the extraordinary properties of certain biomacromolecules, and introduce new functionalities to surfaces. Mucins are versatile glycoproteins that have potential as new building blocks for biomaterial surface coatings. Multilayer films have mostly been assembled through the electrostatic pairing of polyelectrolytes, which results in limited pH and salt stability, and screens charges otherwise available for useful payload binding.

Described herein is the assembly of mucin multilayers that differ from conventional paired polyelectrolyte assemblies. As shown herein, highly stable and functional surface modifications were obtained. Using the lectin Wheat Germ Agglutinin (WGA) to crosslink (which references non-covalent linkage between the lectin and mucins, e.g. glycosylated polymer) mucin-bound sugar residues, it is shown herein that (Mucin/WGA) multilayers can grow into highly hydrated films and sustain exceptional resistance to extreme salt conditions, and a large range of pH. Furthermore, shown herein is that the addition of soluble N-Acetyl-D-Glucosamine can induce the controlled release of WGA from (Mucin/WGA) multilayers. Also shown is that (Mucin/WGA) multilayers can repeatedly incorporate and release a positively charged model cargo. The lubricating, hydrating, barrier and antimicrobial properties of mucins provide multiple applicative perspectives for these highly stable mucin-based multilayer films.

Specifically, described herein is the use of the WGA lectin to crosslink mucin layers. Also described here are the physico-chemical properties of these multilayers which include thickness, level of hydration, morphology, robustness toward ionic strength, and their capacity to bind potential cargo molecules.

Accordingly, in one aspect, the invention is directed to a multilayer film comprising alternating layers of a “glycosylated polymer” (e.g., mucin (e.g., porcine gastric mucin, bovine submaxillary mucin (BSM)), chondriotin sulfate, glycogen in combination with concanavalin A, or a combination thereof) and a lectin (e.g., wheat germ agglutinin (WGA), jacalin or a combination thereof) wherein the lectin crosslinks the glycosylated polymers.

“Mucin” and the like is a highly glycosylated protein capable of forming gels, generally comprising an amino and/or carboxy regions that are cysteine-rich and a central region enriched for serine and/or threonine residues and associated O-linked and/or N-linked oligosaccharides. Exemplary mucins include, for example, certain human mucins such as MUC1 (human GeneID No. 4582), MUC2 (human GeneID No. 4583), MUC5AC (human GeneID No. 4586), and MUC5B (human GeneID No. 727897). In certain embodiments, the mucin is a MUC5AC mucin (see, e.g. UniGene IDs 3881294, 1370646, 1774723, 1133368 and HomoloGene 130646), a MUC5B (see, e.g., HomoloGene 124413), a MUC6 (see, e.g., HomoloGene 18768), MUC2 (see, e.g., HomoloGene 130504, 131905, 132025, or 133451) or combinations thereof. In some particular embodiments, the mucin is a secreted mucin, such as MUC5AC, MUC5B, MUC6, and MUC2. In more particular embodiments, the mucin is a gastric mucin, such as MUC5AC, such as a porcine MUC5AC (see, e.g., UnigeneIDs 441382, 5878683; GeneID No. 100170143, and reference sequences AAC48526, AAD19833, and AAD19832). Other mucins suitable for use concordant with the invention include bovine submaxillary mucin (BSM, also known as MUC19; see e.g. GeneID No. 100140959; see HomoloGeneID 130967; see reference protein sequence XP_{—}003586112.1). A mucin-containing composition provided by the invention can be a mixture of one or more mucins (e.g., at least 2, 3, 4, 5, or more different mucins) and, optionally, may be made up of equal or unequal proportions of the different mucins—e.g., a particular mucin may, in certain embodiments, make up at least about 20, 30, 40, 50, 60, 65, 70, 75, 80, 85, 90, 95, 96, 97, 98, 99, or 100% of the mucins in the composition. Preferably, isolated or purified mucin comprises at least about 50%, 75%, 80%, 90%, 95%, 98% or 99% (on a molar basis) of all macromolecular species present.

Any of the individual mucin sequences described in the above annotations can be adapted for use in the invention, as well as variants thereof, e.g., sequences at least 50, 55, 60, 65, 70, 75, 80, 85, 90, 91, 92, 93, 94, 9, 96, 97, 98, 99, or 100% identical to a functional fragment thereof (e.g., comprising about 40, 50, 60, 70, 75, 80, 85, 90, 95, or 100% of the full length of the mature proteins) that is capable of forming a stable mucin surface. Functional variants will generally preserve the function of the conserved domains present in mucins, including one or more of a cyctine-rich domain, VWC (c102515), GHB-like (c100070), TIL (pfam01826) Mucin2_WxxW (pfam13330), VWD (c102516), c8 (c107383), and FA58C (c112042) domains.

Mucins for use in the invention can be chemically or recombinantly (e.g. in CHO or COS cells) synthesized or isolated from a natural source, e.g., from non-human animals. The mucin can be obtained and purified using the methods described herein from any non-human mammal such as a non-human primate, a bovine, a porcine, a canine, a feline, an equine and the like. In a particular aspect, the non-human gastric mucin is porcine gastric mucin. Porcine gastric mucin can be isolated by the methods described in Celli, J., et al., Biomacromolecules 2005, 6(3), 1329-1333, incorporated by reference in its entirety, preferably omitting the cesium density gradient centrifugation.

“Lectin,” and the like, refers to physiologically compatible protein domains that bind to a sugar moiety associated with a glycosylated polymer. Exemplary lectins for use in the invention include a fragment of wheat germ agglutinin (WGA) lectin (for example, as sold by Vector Laboratories under Catalog No. L-1020) as well as a fragment of the WGA provided in UniprotID P10969 (“P10969”), incorporated by reference in its entirety, (see also GenBank accession no: AAA34257, incorporated by reference, including reference annotations), as well as Jacalin lectin (GenBank accession nos: AAA32680-AAA32677, incorporated by reference) or Sambucus nigra lectin (GenBank accession nos: AAL04122-AAL04119, AAC15885, AAN86132, and AAN86131, incorporated by reference). In particular embodiments, the lectin comprises an amino acid sequence that is at least 60, 65, 70, 75, 80, 85, 90, 95, 96, 97, 98, 99, or 100% identical to P10969 or a functional fragment thereof, such as a contiguous fragment of 30, 40, 50, 60, 70, 80, 90, 100, 120, 140, 160, or 180 amino acids, or more of P10969, e.g., comprising a 30-50 amino acid fragment at least 80% identical to a lectin domain such as a ChtBD1 (PSSM ID: 211512) domain, as exemplified by, for example, any one of the regions defined by amino acids 45-81, 88-124, or 132-167 of P10969, e.g., comprising at least 1, 2, or all 3 of these domains, or higher-order numbers of lectin domains, e.g., polypeptides comprising 1, 2, 3, 4, 5, 6, 7, 8, 9, 10, or more lectin domains.

By way of example, wheat germ agglutinin (WGA) is a 36,000 molecular weight protein consisting of two identical subunits. WGA contains a group of closely related isolectins, with an isoelectric point about pH 9. The receptor sugar for WGA is N-acetylglucosamine, with preferential binding to dimers and trimers of this sugar. WGA can bind oligosaccharides containing terminal N-acetylglucosamine or chitobiose, structures which are common to many serum and membrane glycoproteins. Bacterial cell wall peptidoglycans, chitin, cartilage glycosaminoglycans and glycolipids can also bind WGA. Native WGA has also been reported to interact with some glycoproteins via sialic acid residues (see succinylated WGA). This lectin has proven useful for the purification of insulin receptors and for neuronal tracing.

Additional molecules that can perform the function of a lectin include antibodies that specifically bind to a glycosylated polymer that, when the antibody binds to a sugar moiety associated with a glycosylated polymer, is a lectin according to the invention. “Antibody” refers to both whole immunoglobulins as well as antigen (i.e. glycosylated polymer)-binding fragments of immunoglobulins that contain an antigen-binding domain comprising at least 3, 4, 5, or 6 complementary determining regions (CDRs). Antibodies can be from any source including human, orangutan, mouse, rat, goat, sheep, rabbit and chicken antibodies, as well as synthetic, engineered antibodies. Antibodies may be polyclonal, monoclonal, monospecific, polyspecific, non-specific, humanized, camelized, single-chain, chimeric, synthetic, recombinant, hybrid, mutated, and CDR-grafted antibodies.

The multilayer film can further comprise one or more additional agents (e.g., nucleic acid, protein (polypeptide such as RFD), label (tag such as avidin (steptavidin), biotin, fluorescent protein (green fluorescent protein)), therapeutic protein or nucleic acid, diagnostic protein or nucleic acid, small molecule, drug, antibody and the like) attached (e.g., bound, covalently bound, adsorbed, absorbed, aggregated and the like) to one or more layers of the multilayer film. For example, in one aspect the one or more additional agents is a positively charged molecule (e.g., a polycationic polymer, a growth factor, an antimicrobial peptide, a drug or a combination thereof) that is bound to one or more layers of the glycosylated polymer.

In another aspect, the one or more additional agents is one or more labels that is attached to one or more layers of the lectin. In a particular aspect, the one or more labels is biotin or avidin.

The multilayer film can further comprise a substrate onto which the multilayer film is deposited. Examples of substrates include a polystyrene surface, a gold covered quartz crystal or a polystyrene covered crystal.

In particular aspect, the multilayer film is biocompatible. The multilayer film can comprise any number of layers. For example, the multilayer film can comprise about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 or more (about 30, 40, 50, 60, 70, 80, 90, 100, 1000, 10,000 et cetera) bilayers of alternating layers of the polymer and the lectin.

In particular aspects, the multilayer film is insensitive (e.g., resistant such as resistant to degradation) to ionic strength conditions (e.g., about 1.0, 1.5, 2.0, 2.5, 3.0, 3.5, 4.0, 4.5, 5.0, 5.5., 6.0 M NaCl concentration, or an equivalent ionic strength). In other aspects, the multilayer the film is insensitive to extreme pH conditions (e.g., acidic (<7.0) and/or alkaline (>7.0) conditions, including extreme ranges of pH, covering about 3, 4, 5, 6, 7, 8, 9, 10 or 11, e.g., pH of 3-11 and subranges thereof, e.g., 4-9, 5-8, 3-6, 3-5, 4-6, 8-11, 8-10, 9-10, et cetera).

The final (last, top) layer can be either a lectin layer or a polymer layer.

In another aspect, the invention is directed to a method of producing a multilayer film comprising alternating deposits of a glycosylated polymer with deposits of a lectin on a substrate. As described herein, the method can further comprise washing the film after deposit of each layer onto the substrate. The method can further comprise producing a multilayer film in which the final layer is a lectin layer or a polymer layer.

The method can further comprise contacting the multilayer film with an agent that competes with the glycosylated polymer for attaching to the lectin. In one aspect, the agent is a ligand of the lectin (e.g., a sugar). The multilayer film is maintained under conditions in which all or a portion of the lectin in a final layer or layers of the multilayer film is released, thereby exposing charged groups of an underlying polymer layer. A multilayer film treated so as to release some of the lectin is a “lectin depleted” multilayer film. Varying levels of lectin depletion are possible, e.g., 0.01, 0.05, 0.1, 0.5, 1.0, 2.0, 3.0, 4.0, 5.0, 10, 15, 20, 25, 30, 35, 40, 45, 50, 60, 70, 80, 90% or more of the lectin in one or more layers of the film may be depleted this way.

In particular aspects, the polymer is porcine gastric mucin, the lectin is wheat germ agglutinin and the ligand is N-Acetyl-D-Glucosamine; or the polymer is bovine submaxillary mucin (BSM), the lectin is jacalin and the ligand is melibiose.

The method can further comprising contacting the multilayer film with one or more additional agents (a first agent, a second agent, etc.) and attaching (e.g., bound, covalently bound, adsorbed, absorbed, aggregated and the like) the one or more agents to one or more layers of the multilayer film. The agent can be, for example, a nucleic acid, protein (polypeptide), label (tag such as avidin (steptavidin), biotin, fluorescent protein (greem fluorescent protein)), therapeutic protein or nucleic acid, diagnostic protein or nucleic acid, small molecule, drug, antibody and the like). As will be appreciated by those of skill in the art, the conditions for attaching an agent to the multilayer film will depend upon the agent to be attached and the layer(s) to which the agent is to be attached. For example, in one aspect the one or more additional agents is a positively charged molecule (e.g., a polycationic polymer, a growth factor, an antimicrobial peptide, a drug or a combination thereof) that is bound to one or more layers of the glycosylated polymer.

In another aspect, the one or more additional agents is one or more labels that is attached to one or more layers of the lectin. In a particular aspect, the one or more labels is biotin or avidin.

The method can further comprise maintaining the multilayer film under conditions in which the one or more agents are released from the multilayer film. Examples of such conditions include altering ionic conditions, pH conditions or a combination thereof.

The method can further comprising contacting the multilayer film with one or more additional agents (a second agent, a third agent, etc.) after the one or more agents are released from the multilayer film and maintaining the multilayer film under conditions in which the one or more additional agents binds to the multilayer film, thereby reloading the multilayer film. The agent can be the same agent as was previously attached to the multilayer film or a different agent.

In a particular aspect, the method can further comprise contacting the film to which biotin is attached to one or more layers of lectin (e.g., the final layer of lectin in the multilayer film) with a composition comprising one or more molecules of streptavidin attached to one or more molecules of interest, thereby producing a combination. The combination is maintained under conditions in which the streptavidin that is attached to the molecule of interest binds to the biotin attached to the one or more layers of the lectin.

In another aspect, the invention is directed to a multilayer film produced as described herein.

As will be appreciated by those of skill in the art, the multilayer films described herein can be used in a variety of ways. For example, the multilayer films can be used to detect or isolate a molecule of interest, for example in a sample (e.g., a biological sample such as blood, urine, lymph, tissue; an environmental sample, such as soil, water, etc.). In one aspect, the invention is directed to a method of detecting or isolating a molecule of interest comprising attaching streptavidin to the molecule of interest. The molecule of interest is contacted with the multilayer film to which biotin is attached to one or more layers of lectin (e.g., the final layer of lectin in the multilayer film), thereby producing a combination. The combination is maintained under conditions in which the biotin of the multilayer film binds to the streptavidin of the molecule of interest, thereby detecting or isolating a molecule of interest.

As described herein, a variety of agents can be attached to the multilayer film (e.g., a biocompatible multilayer film) and moreover, released from the multilayer film under conditions which will not degrade the multilayer film. Thus, the multilayer films can also be used to deliver a molecule of interest, for example, to an individual (e.g. a subject) in need thereof (a therapeutic agent, a diagnostic agent, an agent that prevents/treats infection (e.g., wound healing). In yet another aspect, the invention is directed to a method of delivering an agent to a subject in need thereof comprising introducing the multilayer film described herein to the subject thereby delivering the agent. A “subject” refers to a mammal, including primates (e.g., humans or monkeys), cows, sheep, goats, horses, dogs, cats, rabbits, guinea pigs, rats, mice or other bovine, ovine, equine, canine, feline, rodent or murine species. Examples of suitable subjects include, but are not limited to, human patients. In particular embodiments, the subject to be treated by the methods provided by the invention is human and can be male or female and may be at any stage of development: e.g., prenatal, neonatal, infant, toddler, grade-school-age, teenage, early adult, middle-age, or geriatric, e.g., at least or about 1, 2, 3, 4, 5, 6, 7, 8, 9, 10, 15, 18, 20, 21, 25, 30, 35, 40, 45, 50, 55, 60, 65, 70, 75, 80, 85, 90, 95, 100, 105 years old, or more. Biological surfaces to which the multilayer films of the invention are provided may either be intact (e.g. healthy) or injured (e.g. by burn, rash, irritation, cut, tear, disease, et cetera).

Thus, the invention is also directed to a pharmaceutical composition comprising one or more multilayer films described herein. That is, the multilayer film can be formulated as part of a dressing for a wound. For instance, the compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. The pharmaceutical compositions of this invention can also be administered as part of a combinatorial therapy with other compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compounds assembled in a blister pack or other dispensing means is preferred. For the purpose of this invention, unit dosage is intended to mean a dosage that is dependent on the individual pharmacodynamics of each compound and administered in FDA approved dosages in standard time courses.

### EXEMPLIFICATION

### Example 1

### Material and Methods

### Materials and Reagents

Pig gastric mucin was purified from pig stomachs as reported in Celli, J., et al., Biomacromolecules 2005, 6, 1329-1333, omitting the cesium density gradient. Glycosylated high molecular weigh protein was recovered and confirmed to be mostly MUC5AC and MUC6 type mucin by mass spectrometry. The lypholized mucin samples were dissolved overnight in ultrapure water at 2 mg/mL at 4° C. Alexa488 labeled WGA was purchased from Invitrogen and reconstituted at 1 mg/mL in ultrapure water. Purified pig gastric mucin was labeled with the Alexa488 dye by mixing 0.1 mg of Alexa488-carboxylic acid succinimidyl ester (Invitrogen) dissolved in DMSO at 10 mg/mL, in 4 mg of mucin dissolved in 0.1 M bicarbonate buffer (pH 9). The mixture was shaken for 1 hour at room temperature, then the pH was lowered to 7, and the sample dialyzed against water for 5 days (MWCO 20 KDa). All other polymers and reagents: Wheat Germ Agglutinin lectin (WGA), Poly-1-lysine (PLL), fluorescein labeled PLL (PLL-FITC) and N-acetyl-D-glucosamine were bought from Sigma (St. Louis, Mo., USA).

### Multilayer Film Buildup

Multilayers were built by alternating depositions of mucin and the lectin WGA from dilute solutions. Mucin was prepared at a concentration of 0.2 mg/mL and lectin at 0.1 mg/mL in buffer containing 20 mM Hepes (pH 7.4) and 0, 200 or 400 mM NaCl. Mucin was left to adsorb to the surface for 15 minutes, lectin for 5 minutes. Between layering, two washes of 5 min were performed with the same buffer as used for buildup. For 96 well plates, 50 μL of mucin and WGA solution, and 1504 of washing solution, was used. For QCM-D experiments, 300 μL of WGA and mucin solution, and 1 mL of washing solution, was used. The resulting multilayers are termed (Mucin/WGA)n, with n being the number of layer pairs.

Quartz Crystal Microbalance with Dissipation Monitoring

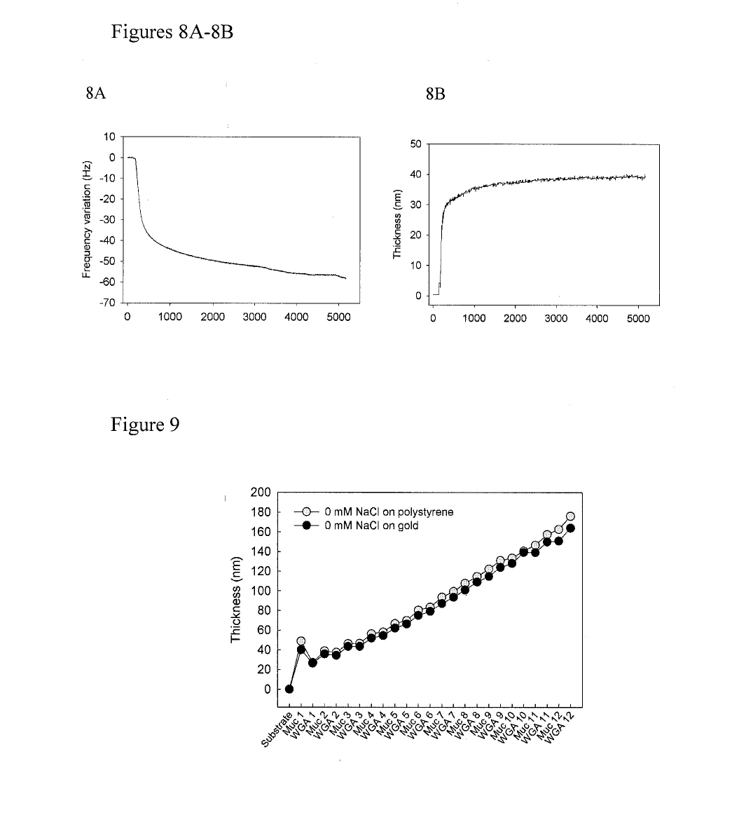

The multilayer buildup was followed by Quartz Crystal Microbalance with Dissipation monitoring (QCM-D, E4 system, Q-Sense, Sweden). The multilayers were grown on a gold covered quartz crystal, cleaned with warm 2% SDS and 0.1 M HCl, rinsed with ultrapure water, and further cleaned by ozone treatment for 15 minutes. Multilayer growth on polystyrene coated crystals was similar as on gold (FIG. 8). The crystal vibration was followed at its fundamental frequency (about 5 Mhz) and the 5 overtones (15, 25, 35, 45, 55 and 65 Mhz). Changes in the resonance frequencies, and in dissipation of the vibration once the excitation is stopped, were followed at the 6 frequencies. As suggested by the high dissipation values (Mucin/WGA) multilayers are highly hydrated and possess viscoelastic properties requiring the measurement data to be modeled. The Voigt based model (Voinova, M., et al., Physica Scripta, 1999, 59, 391) (i.e., a spring and dashpot in parallel under no slip conditions) was used to calculate the hydrated thickness, assuming a density of 1050 g·m⁻³ (Weber, N, et al., J Biomed Mat Res Part A, 2005, 72A, 420-427) and that the multilayer is homogeneous in thickness and over the crystal's surface. The multilayers were built up to 12 layer pairs, except for (Mucin/PLL) for which a loss in signal occurred for most frequencies after 8 layer pairs.

### Dry Thickness of the Multilayer Films

Dry thickness was determined by spectroscopic ellipsometry. This surface sensitive optical technique allows thin film thickness and optical properties to be determined. These parameters are determined by recording changes of light polarization at a fixed angle of incidence)(70° and various wavelengths upon reflection on the dried multilayer film. The multilayers assembled on QCM-D crystals were rinsed with water and thoroughly dried with a flow of nitrogen and spectroscopic scans performed (XLS-100, J.A. Woollam Co., Lincoln, Nebr. USA) with 70 scans per measurements, and 4 measurements per sample. The data was modeled use the WVASE32 software (version 3.768) and assuming a multilayer model composted of a known Si substrate (0.2 mm), a gold layer (75 nm) and a Cauchy layer of unknown thickness and optical properties corresponding to the (Mucin/WGA) multilayer. The mass of the dry multilayer was then estimated by assuming a density of 1200 g·m⁻³ as previously measured for other similar systems (Caruso, F., et al., Langmuir 1998, 4559-4565).

### Multilayer Film Imaging

For fluorescence microscopy observations, the multilayer was built using the fluorescent mucin-Alexa488 conjugate, in untreated polystyrene 96 well plates with optically thin bottoms (Costar 3615 Corning, Corning N.Y., USA). The multilayers were scratched with a pipette tip and imaged using an Observer Z1 inverted fluorescent microscope (Zeiss, Oberkochen, Germany) and a 10×0.3 NA or 100×1.4 NA lens (Zeiss). For AFM imaging, the multilayers were built on QCM-D gold-covered crystals, dried with a flow of dry nitrogen gas, and observed in contact mode using pyramidal cantilevers (NP-S10, Veeco, Santa Barbara, Calif., USA) and a Nanscope IVAFM (Veeco, Santa Barbara, Calif., USA).

### Multilayer Film Composition, Resistance to Extreme Conditions, and WGA Release

The composition of the (Mucin/WGA) multilayer films was obtained by assembling the film in untreated polystyrene 96 well plates (Falcon 351172) using either fluorescently labeled mucin (40% of labeled mucin) or fluorescently labeled WGA (10% of labeled WGA) as tracers. These multilayers were built in 0 mM NaCl in 20 mM Hepes buffer (pH 7.4). The fluorescence of each well was measured after each new layer using a fluorescence plate reader (Spectramax M3, Molecular Device). Mucin and lectin were quantified by calibrating fluorescence in solution with known amounts of mucin. WGA was released from the mucin capped multilayers by adding 200 μL of 100 mM N-acetyl-D-glucosamine solution in each well. For low pH treatment, 200 μL of acetate buffer (0.1 M, pH 3) was used. The high pH treatment consisted of 200 μL carbonate buffer (0.1 M sodium carbonate and 0.1 M sodium bicarbonate, adjusted to pH 9) or KCl/NaOH buffer (0.1 M, pH 12). All multilayer films were washed 3 times with 150 μL of 20 mM Hepes buffer (pH 7.4) after treatment. Compositional changes in (Mucin/WGA)₁₂ multilayers were measured by comparing the total fluorescence before and after these treatments, relative to the fluorescence of non-treated wells.

### PLL Incorporation in (Mucin/WGA) Multilayer Films

To investigate the capacity of (Mucin/WGA) multilayer films to incorporate positively charged molecules, 404 of a 0.5 mg/mL solution of PLL-FITC was deposited on (Mucin/WGA)₁₂ or (Mucin/WGA)_{11.5} multilayers which were untreated, or treated with GlcNAc to release WGA. After 30 minutes, the PLL-FITC solution was removed, and the wells were washed 5 times with 150 μL of 20 mM Hepes solution (pH 7.4) before fluorescence was quantified. Fluorescence was converted to mass of PLL by calibration in solution.

### Multilayer Film Cytotoxicity

The epithelial HeLa cell line was grown up to 70% confluency in T25 flasks with DMEM media supplemented with 10% fetal bovine Serum (Invitrogen) and 1% antibiotics (25 U/mL penicillin, 25 μg/mL streptomycin (Invitrogen). The cells were detached using trypsin-EDTA (Invitrogen). The detached cells were washed to remove the trypsin before being plated at a density of 27,000 cells/cm² on the multilayer constructed in the wells of 96 well plates. The cells were incubated at 37° C., 5% CO₂ under humidified atmosphere for 24 hours before being stained with the live dead stain (2 μM calcein and 2 μM Ethidium homodimer-1 in DMED media, Invitrogen) for 20 minutes. Images were acquired on an Axio Observer Z1 microscope (Zeiss, Oberkochen, Germany) using an EC-Plan Neofluar 10×0.3 NA lens (Zeiss). Live cells (green) and dead cells (red) were counted counts with the image analysis software ImageJ using the cell count plug-in.

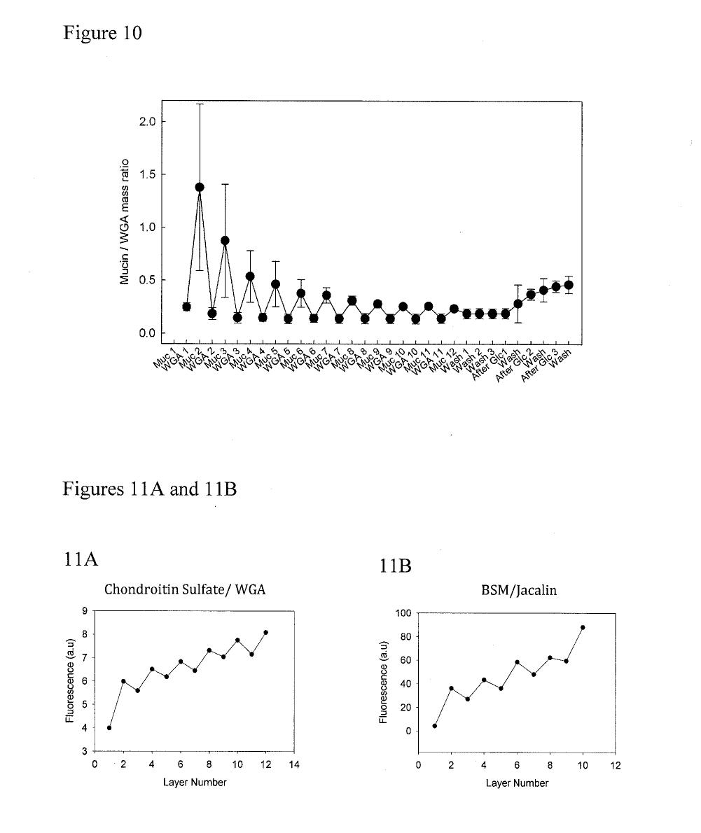

FIGS. 8-10 show QCM-D data showing that mucin cannot spontanously form multilayer films in the conditions used. The QCM-D data for the growth of the multilayers on both gold and polystyrene covered surfaces is also shown.

### Results and Discussion

### Mucin Multilayers can be Cross-Linked Via Lectins

When a dilute solution of mucins is subjected to the substrate of a Quartz Crystal Microbalance with Dissipation monitoring (QCM-D), a mucin coating is formed. Previous reports demonstrated that such first mucin coating forms as a 40-60 nm thick and hydrated monolayer. In an attempt to generate a mucin multilayer it was observed that this first coating was repulsive towards further mucin molecules, indicating that mucin multilayers cannot easily be auto-assembled (FIG. 8). This is in agreement with previous AFM measurements, which indicate the adsorbed mucin will repel individual mucins and other negatively charged polymers by steric and electrostatic forces.

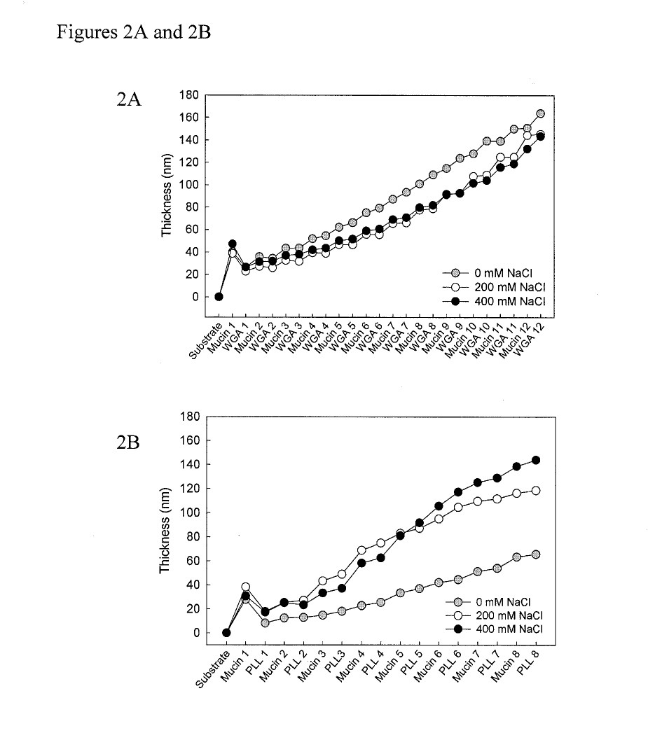

Next, whether a layer-by-layer buildup of mucins could be achieved by cross-linking individual mucin layers with lectins was tested. In this experiment, the deposition of mucins was alternated with the deposition of WGA, with washing steps in-between. The QCM-D measurements were modeled to obtain the hydrated thickness of the multilayers as the mucin and lectin layers were deposited. FIG. 2A shows that the initial mucin layer decreased in thickness as the first lectin layer was deposited, possibly due to a collapse of the glycan chains protruding from the mucins. Then, for the 12 subsequent mucins-WGA layer pairs, an almost linear growth was observed (FIG. 2A), indicating that mucins and WGA can indeed auto-assemble into multilayer films. It appeared that the lectins were able to cross-link soluble mucin polymers onto already existing mucin layers, overcoming the electrostatic and sterical repulsions between soluble and adsorbed mucins.

To analyze the assembly process of (Mucin/WGA) multilayers further, they were constructed at three different ionic strengths, using 0, 200 and 400 mM NaCl added to the buffer solution. Strikingly, no major difference in the final thickness of the multilayers could be measured. For comparison, the growth of multilayers composed of mucins and the positively charged Poly-L-lysine (PLL) polymer was sensitive to salt (FIG. 2B), resulting in an about 2-fold difference in the final thickness with no salt and 200 mM NaCl, respectively. The relative insensitivity of (Mucin/WGA) multilayers growth toward ambient ionic strength indicated that specific lectin/sugar interactions were driving the multilayer's assembly, as opposed to electrostatic interactions between surface charges of both mucin and WGA. These specific lectin/sugar interactions that generated the multilayer seemed to differ mechanistically from the ionic pairing occurring in (Mucin/PLL) multilayers. Indeed, in the case of polyelectrolyte-based assemblies, higher salt concentrations increased the charge shielding and effectively changed the number of charged groups available for ionic paring, thus influencing the multilayer's growth.

Note that the growth curves of the mucin-WGA multilayers were rather linear. Weak interactions between the components usually result in exponentially growing multilayers, as the polymers can diffuse to the surface and provide additional binding sites for the next layer. Strong interactions will result in tightly stacked polymers, linearly increasing the thickness at each deposition step. The linear character of the mucin-WGA multilayer growth therefore indicates that interactions were strong between the components and that diffusion of the components within the assembly was limited.

### (Mucin/WGA) Multilayers are Highly Hydrated

For further characterization, the (Mucin/WGA) multilayers were assembled in Hepes buffer without NaCl, since the assembly process appears independent of ionic strength. The (mucin/WGA)₁₂ multilayers assembled on both the gold surface used for AFM measurement and on the polystyrene surface when analyzed by light microscopy. No differences in thickness or growth curve were measured on these two surfaces by QCM-D (FIG. 9), indicating that the initial mucin layer was able to mask variations in surfaces properties from the rest of the multilayer.

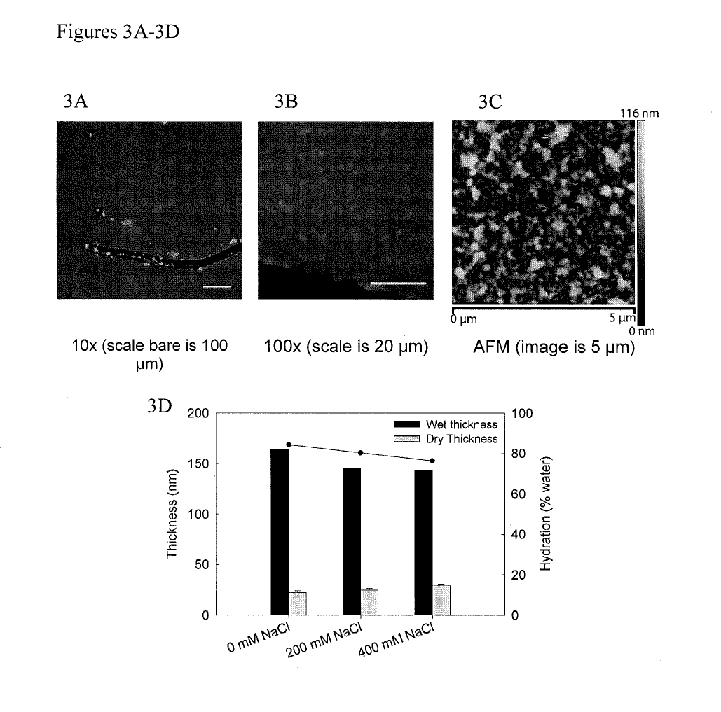

The morphology of the multilayer was characterized both by fluorescence microscopy, using fluorescently labeled mucins, and Atomic Force Microscopy (AFM). At low (10×) magnification the multilayers appeared rather homogeneous (FIG. 3A). However, at 100× magnification, aggregates in the micrometer range could be visualized (FIG. 3B). AFM measurement on dried multilayers revealed these 1-3 μm aggregates in more detail (FIG. 3C). The small aggregates detectable at higher magnification by light microscopy and by AFM could come from the tendency of mucin molecules to auto-assemble into multimeric structures.

The hydration (or swelling) of many polyeletrolyte multilayers can be modulated by changing the salt concentration, as the degree of charge screening can modulate the density of polymer packing, and water-filled volumes. FIG. 3C shows that the level of hydration for (Mucin/WGA)₁₂ multilayers was independent of the salt concentration and consisted of more than 80% water, which is comparable to previous measurements on mucin- and other biopolymer-based multilayers. Thus, mucins appeared to maintain their native water binding capacity when arranged as multilayers. Here, the independence of hydration towards salt concentration further supports that the multilayer's assembly is largely independent of ionic strength.

### (Mucin/WGA) Multilayers are Exceptionally Resistant to High Salt Concentrations

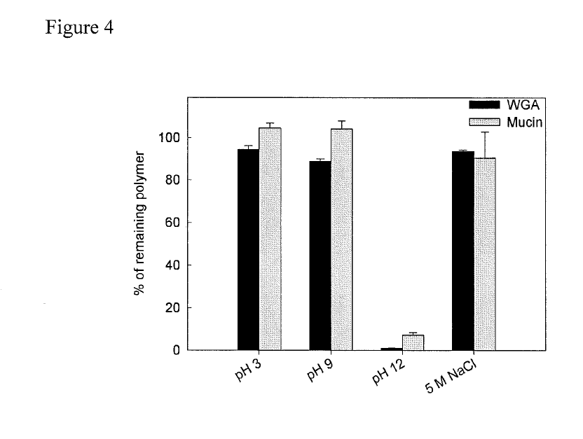

Since mucin multilayers are insensitive toward ionic strength during assembly, it was postulated that they might also be resistant toward high ionic strength, and possibly extreme pH conditions, after buildup. To test this possibility (Mucin/WGA)₁₂ multilayers were thus built in 0 mM NaCl at pH 7.4, and then subjected to 5 M NaCl, pH 3, pH 9, or pH 12. Indeed, the multilayers showed exceptional resistance to these extreme conditions, and neither mucin nor WGA content decreased by more than 20%. Only a pH of 12 dismantled the structure of the multilayer (FIG. 4).

Many other electrostatically based multilayers, for example (Mucin/Chitosan), dismantle readily when exposed to different pH or ionic strengths than the condition used for the buildup. Several polyelectrolyte multilayer systems can resist pH changes within the range tested here, however resistance to 5 M NaCl is unusual. It is possible that this effect is due to the exceptionally high local concentrations of both lectin and lectin binding sites within the multilayer that result in WGA-carbohydrate interactions with high avidity.

Controlled Release of WGA from (Mucin/WGA) Multilayers by N-Acetyl-D-Glucosamine

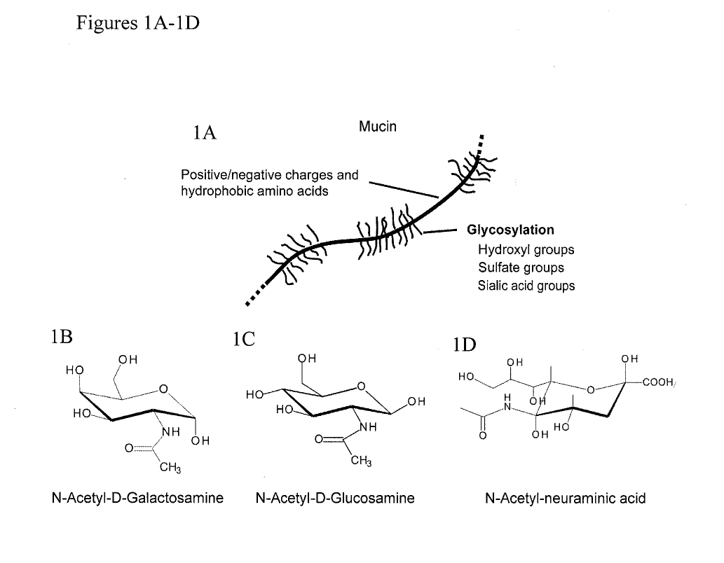

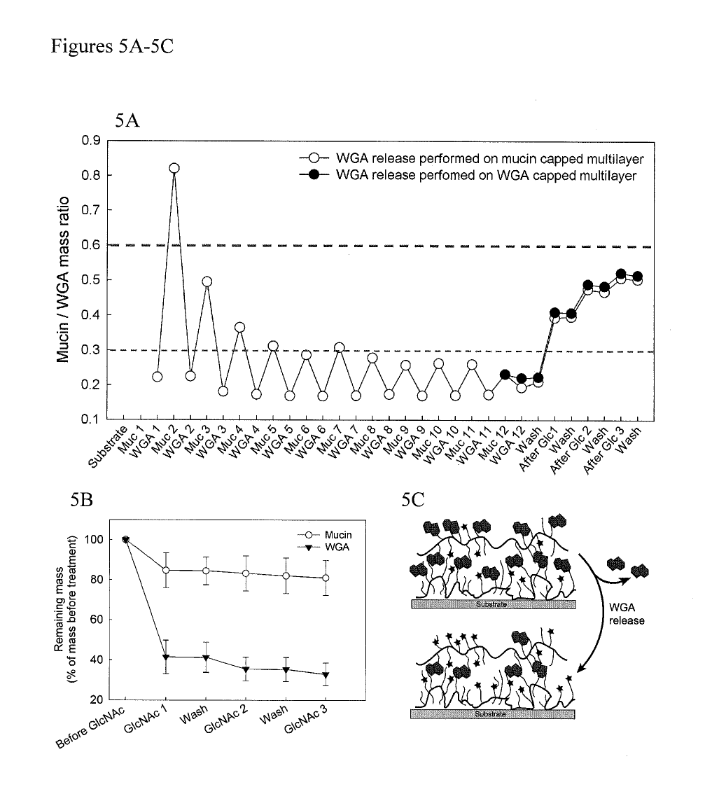

If strong ionic strength does not destabilize the multilayer, perhaps a specific competition for the lectin's binding sites can. To test this possibility, (Mucin/WGA)₁₂ multilayers were subjected to N-Acetyl-D-Glucosamine (GlcNAc), a known ligand for WGA (FIG. 1C). The composition of the multilayer was followed as it was assembled, revealing that the mucin to WGA weight ratio was around 0.2. After assembling a 12 layer pair, GlcNAc was added, effectively raising the ratio to 0.5 (FIG. 5A) Changing the last layer from WGA to mucin only slightly increased the ratio. The WGA content of the assembly decreased by about 85% after GlcNAc was added. In contrast, the amount of mucin only dropped by 20% after three consecutive treatments with GlcNAc (FIG. 5B).

Structural and compositional changes in 3D hydrogels and multilayers through competition-induced release of lectins have been demonstrated at several occasions, mostly in the context of auto-regulated insulin delivery systems. The specific benefit of this system is that the mucins were stably anchored within the assembly, therefore, one can induce a relative enrichment in mucin of the multilayer by releasing the WGA. It is likely that the WGA molecules interacting with a lower number of mucin sugar residues will be preferentially released over WGA molecules in which all 8 sugar-binding sites are engaged in cross-linking (FIG. 5C).

### (Mucin/WGA) Multilayers can Repeatedly Incorporate and Release the Positively Charged Polymer PLL

The potential to release mucin-bound WGA is of great interest in the context of drug delivery, where WGA-tethered molecules could be specifically released in the presence of sugars. But more importantly, the release of WGA uncovers charged groups like sialic acids, which are otherwise sequestered by the lectin. A controlled release of WGA from the mucins could likely be used to adjust the binding capacity of positively charged molecules, such as polycationic polymers, growth factors, antimicrobial peptides, and certain small drugs, within the multilayer (FIG. 5C).

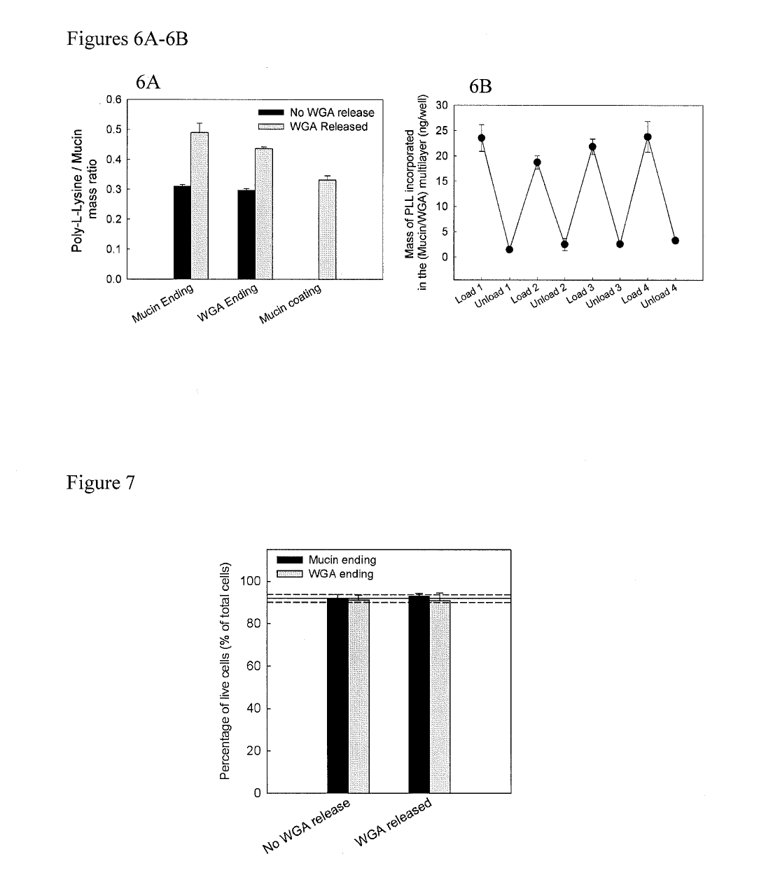

To test if (Mucin/WGA)₁₂ multilayers can in principle be loaded with positively charged molecules such as drugs, Poly-L-Lysine (PLL) was used to analyze its ability to incorporate into the (Mucin/WGA) multilayers. PLL is a positively charged polypeptide that was used here as a model for positively charged biomacromolecules of interest. FIG. 6A depicts that mucin multilayers can efficiently incorporate PLL; the mass ratio of PLL to mucin was similar for a single coating as for a multilayer prior to WGA release. In the next step it was asked if the partial release of WGA would affect PLL loading and indeed, the PLL to mucin ratio increased to almost 0.5 (FIG. 6A). This revealed that (Mucin/WGA)₁₂ multilayers can be loaded with PLL and that the partial dissociation of WGA further increased its binding capacity. This effect was likely caused by an increased availability of mucin-bound negative charges after WGA release, and potentially also by the reduction of positive charges introduced by WGA (Ip=9). This indicates that using lectins to form mucin multilayer films is advantageous to tune the loading of potential cargo molecules into mucin multilayers.

An additional strength of the mucin-multilayer for drug delivery applications is its resistance to high ionic strength. PLL can be released at 5 M NaCl (FIG. 6B), and since the mucin multilayer remains intact in these conditions, the same mucin multilayer can be re-charged with PLL four times with no loss of PLL binding capacity (FIG. 6B).

Here, the loading of mucin multilayers with model positively charged polymers was studied, however, cells could engage in specific interactions with the mucins. In particular sialic acid is recognized as a ligand with high biological significance. It is likely that modulating the release of WGA can tune the availability of this type of binding site, thereby influencing the interaction of certain cells with the multilayer. In addition, the mucus barrier hosts many bioactive molecules such as growth factors and antimicrobial peptides in vivo. Thus, mucin-based coatings with complexed bioactive molecules for biomedical applications such as drug delivery, wound healing, and antimicrobial surfaces are also encompassed herein.

### (Mucin/WGA) Multilayers are Non Cytotoxic

In the context of biomedical applications, mucin multilayers would be placed in contact with cells, and must thus prove to be non-cytotoxic. Previous studies reported a certain toxicity of lectin towards cancer cell lines, for example. Therefore, the cytotoxicity of (Mucin/WGA)₁₂ multilayers toward epithelial HeLa cells was tested. After 24 hours on both mucin or WGA-capped multilayers, the cells remained as viable as on standard polystyrene surfaces as judged by a fluorescence-based live/dead assay (FIG. 7). Releasing the WGA with GlcNAc before seeding the cells did not affect viabiliy.

Many reports of lectin containing constructs for biomedical application exist, and although lectins are part of our diet, there is still uncertainty regarding the cytotoxicity of lectins. In the mucin-multilayers used here, the cytotoxicity of WGA appears to be suppressed by its complexation with mucins. It is likely that binding of WGA to mucins reduces its ability to cell penetrate into the cells. WGA is cytotoxic only when uptaken by the cell, thus, sequestration by mucins should limit its toxicity. Moreover, the mucin multilayers presented here are stable, releasing little of none of its components into solution, limiting potential cytotoxicity. This is in contrast of other polysaccharide-based multilayers, which partially disassemble into solution and drastically influence cell behavior if not covalently cross-linked.

### CONCLUSIONS

In conclusion, provided herein is a biopolymer-based multilayer film made from mucins and the lectin WGA. This multilayer is resistant to extreme NaCl concentrations, and a wide range of pH. We also show that such mucin multilayers can undergo multiple rounds of loading and release of a model substrate, PLL, without disintegrating. The data provided herein emphasizes at least two points. First, using lectin/sugar interactions may be an interesting strategy to overcome the often-noticed sensitivity of polyelectrolyte multilayers toward changes of pH and ionic strength. This strategy likely waives the need to covalently cross-link individual layers within the multilayer before in vivo use, which is often achieved with cytotoxic chemicals, and can result in inactivation of incorporated functional molecules. The large variety of lectins that are commercially available, each with different binding specificities, opens multiple possibilities for new lectin/polysaccharide systems. Second, a method to assemble mucin-based multilayer films has been developed. This system can be used, for example, to generate mucin-based surfaces for a range of biomedical applications. If the in vivo properties of mucins are preserved in the multilayer, it might be possible to create new types of surface coatings for lubrication, selective drug delivery, and anti-fouling.

### Wheat Germ Agglutinin Physic-Chemical Properties

### Wheat Germ Agglutinin

Protein type: Globular protein
Sub-unit number: 2

### Molecular Weight: 36 Kda

Isolectric point (Ip): 9
Binding site number: 8
Provided above are the main chemical-physical properties of the Wheat Germ Agglutinin lectin.

### Example 2

### Generalization of the Technique to Other Sugars/Lectin Couples

Multilayers films can be built using other sugar-containing polymers or polysaccharides. Provided herein is data on film building using Chondroitin Sulfate and the Wheat Germ Agglutinin lectin (WGA) as well as with Bovine Submaxillary Mucin (BSM) couples with the Jacalin lectin.

The data shows the total fluorescence of films built using fluorescently labeled WGA or Jacalin. This is direct proof that there is accumulation of material as the number of layer is increased (FIG. 11).

Additional work has been done towards using these films as sugar sensitive functionalizable surfaces. For this work film made from BSM and Jacalin were used.

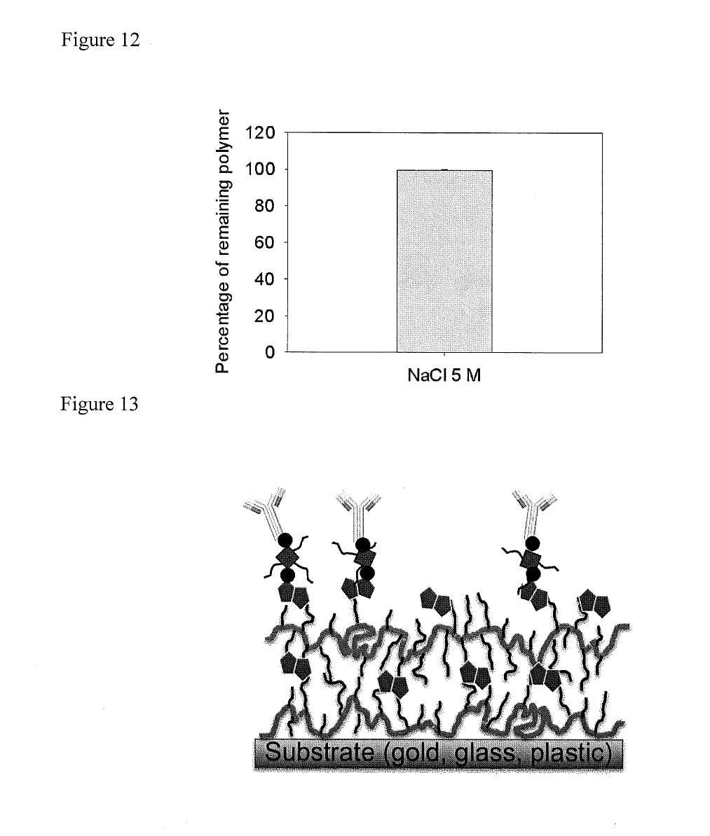

These films buildup using the same method as for the (Pig Gastric Mucin/WGA) films described in Example 1. They were also very resistant, at least to high salt concentrations (FIG. 12).

In the schematic of FIG. 13, the BSM is in the solid wavy horizontal lines, the pentagons are the jacalin lectin, the circles are biotin molecules, the squares (diamonds) are avidin molecules, and the “Y” shaped molecules are a molecule of interest.

In this particular case, on the last layer of the (BSM/jacalin) film, Jacalin-biotin is used. Avidin can then bind to that jacalin-biotin and can then bind to any biotinylated molecules of interest.

One can then vary the loaded amounts by changing the concentration of biotinylated molecules of interest used or by changing the number of layers for which biotinylated Jacalin is used as described herein. This proof of principal was investigated using the fluorescent biotin-fitc (instead of a biotinylated molecules of interest).

Thus loading and sugar-related release was determined by measuring variation of fluorescence of the films (FIG. 13).

### Incorporation of Biotin-Fitc:

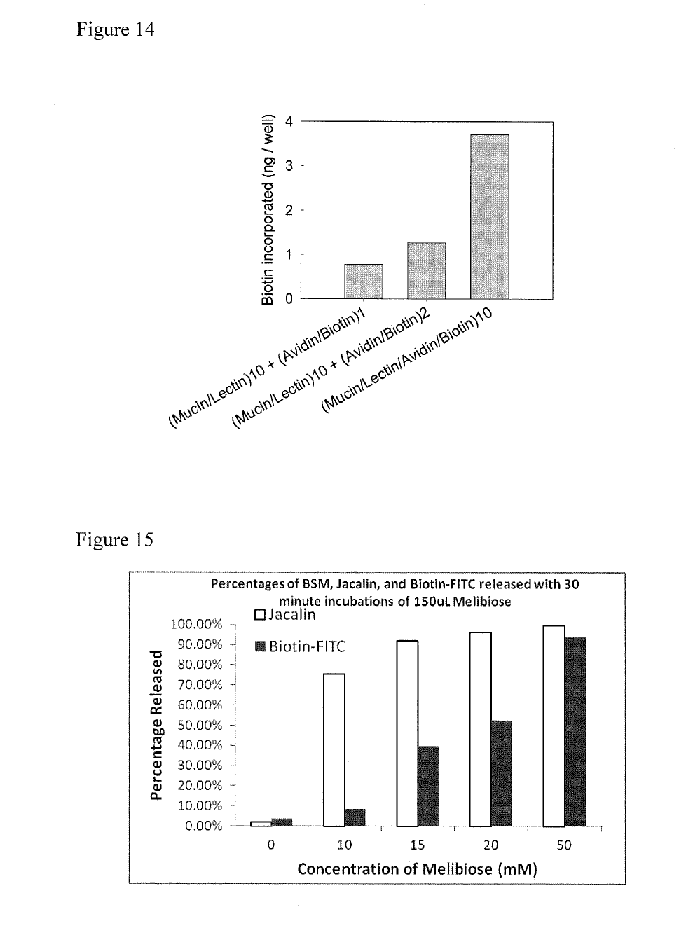

Variation of the loaded amount of the molecule of interest (here, biotin-fitc) can be achieved by variation of the number of layer comprising jacalin-biotin. The data presented in FIG. 14 show amounts of biotin-fitc incorporated in the case of 1, 2 or all 11 layers comprising the (Jacalin-biotin/Avidin/Biotin-fitc) sequence (FIG. 14).

The system is thus highly tunable in the amounts of molecule of interest than can be incorporated. Also the localization of the molecule of interest within the films could be controlled. It can be places on the top, in the middles or at the bottom.

### Sugar-Induced Release

Melibiose, a small sugar, efficiently competed for the Jacalin/BSM interaction. Introducing a solution of melibiose over the films induced the destruction of the film.

This first piece of data shows how the film has been deconstructed as a function of melibiose concentration. The data was obtained by building films with fluorescently labeled BSM and jacalin and following the drop in fluorescence of the films after exposure to melibiose. These drops in fluorescence corresponded to the deconstructions of the film (FIG. 15).

The results show that jacalin was more easily removed then BSM in the film. As the concentration of sugars was increased more and more of the films were deconstructed.

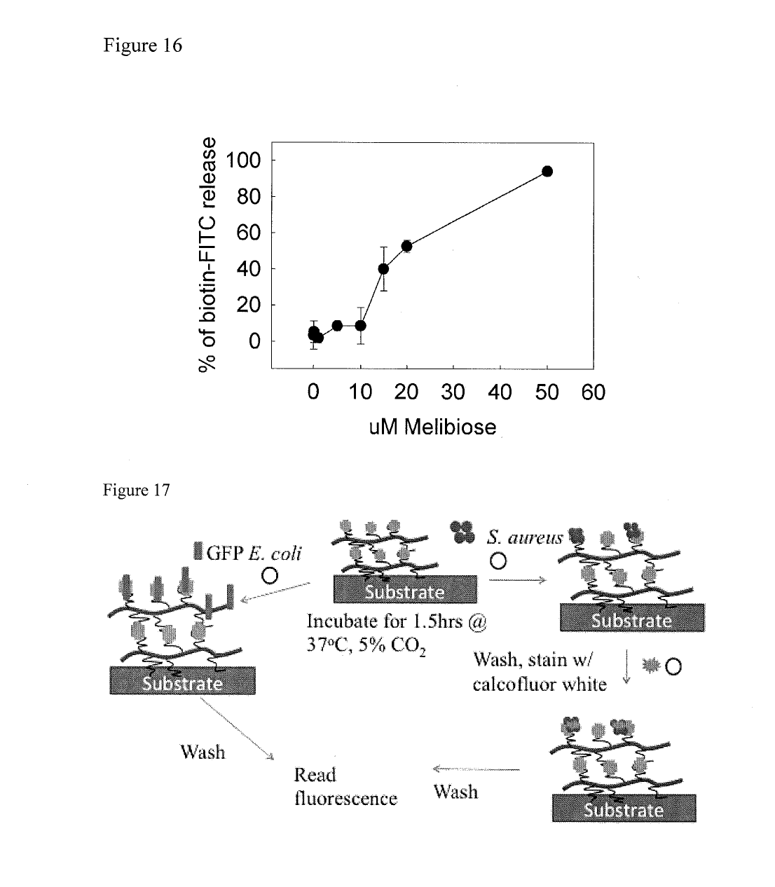

Linked to these results are the release of the molecule of interest as a function of sugar concentration. Shown herein is that various percentage of the incorporated amount can be released by variation of the sugars concentration (FIG. 16).

### Example 3

### Bacteria Interaction

We wanted to test the ability of the mucin multilayer to prevent adhesion of two types of bacteria: Escherichia coli and Staphylococcus aureus. To test for antimicrobial activity, we carried out a study where 12 bilayers of mucin/lectin were built with either a mucin terminal surface or a lectin terminal surface. The multilayers were challenged with GFP transformed Escherichia coli or Staphylococcus aureus, incubated for one and a half hours and subsequently washed. Surfaces challenged with S. aureus were stained with calcofluor white and washed. Fluorescence readings were taken to quantify the amount of bacteria adhered to the surfaces. A schematic of these experiments is shown in FIG. 17.

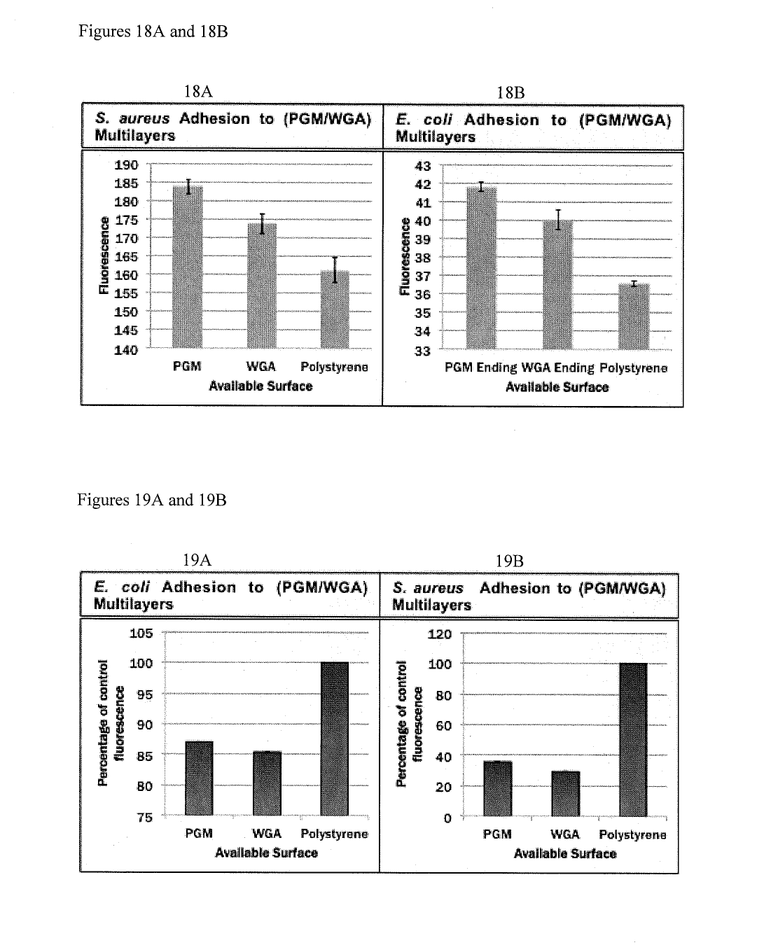

For films made of Wheat Germ Agglutinin lectin and Pig Gastric Mucin (WGA/PGM), both E. Coli and S. aureus tend to bind to the film better than to polystyrene, which is a surface known to favor binding of these bacteria. We hypothesized that this was due to the WGA lectin binding the bacteria and favoring their anchorage to the surface. The lectins can be released from the film using a sugar solution. Here, a solution containing 100 mM N-Acetyl-D-Glucosamine was used to release the WGA from the film After the WGA release, the adhesion of the bacteria to the film decreased dramatically. The films were now less adhesive that the polystyrene surface. FIGS. 18A, 18B, 19A, and 19B illustrate these results, with FIG. 18 showing results before lectin depletion and FIG. 19 showing results after lectin depletion.

By controlling the lectin content of the multilayer films, we were able to tune the adhesion of bacteria to the surface of the material. On can imagine developing surface that are adherent to bacteria and releasing these bacteria at-will by applying a sugar solution that triggers the lectin release. This type of surface could be used for diagnostics, for example.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs, Unigene IDs, or HomoloGene ID, or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g. elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for materials that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus to the extent an element is described in one or more references known to the person having ordinary skill in the art, they may be excluded from the claimed invention by, inter alia, a negative proviso or disclaimer of the feature or combination of features.

While this invention has been particularly shown and described with references to example embodiments thereof, it will be understood by those skilled in the art that various changes in form and details may be made therein without departing from the scope of the invention encompassed by the appended claims.

1. A multilayer film comprising alternating layers of a glycosylated polymer and a lectin, wherein the lectin crosslinks the glycosylated polymers.

2. The multilayer film of claim 1, wherein the glycosylated polymer is mucin, chondriotin sulfate, glycogenin in combination with concanavalin A, or a combination thereof.

3. The multilayer film of claim 2, wherein the mucin is porcine gastric mucin, bovine submaxillary mucin (BSM) or a combination thereof.

4. (canceled)

5. The multilayer film of claim 1, wherein the lectin is wheat germ agglutinin (WGA), jacalin or a combination thereof.

6. The multilayer film of claim 1, further comprising one or more additional agents attached to one or more layers of the multilayer film.

7-10. (canceled)

11. The multilayer film of claim 1, further comprising a substrate onto which the multilayer film is deposited.

12. (canceled)

13. (canceled)

14. The multilayer film of claim 1, comprising about 2, 3, 4, 5, 6, 7, 8, 9, 10, 11, 12, 13, 14, 15, 16, 17, 18, 19, 20 or more bilayers of alternating layers of the polymer and the lectin.

15-18. (canceled)

19. The multilayer film of claim 1, wherein the final layer is a lectin layer.

20. The multilayer film of claim 1, wherein the final layer is a polymer layer.

21-26. (canceled)

27. The multilayer film of claim 1, which is lectin depleted.

28-30. (canceled)

31. A pharmaceutical composition comprising the multilayer film of claim 1.

32. A method of producing a multilayer film, comprising alternately depositing a glycosylated polymer and depositing a lectin on a substrate.

33-35. (canceled)

36. The method of claim 32, wherein the polymer is a mucin and is applied at a concentration of about 0.1 mg/mL to about 2.0 mg/mL.

37. (canceled)

38. The method of claim 32, wherein the lectin is applied at a concentration of about 0.05 to about 2.0 mg/mL.

39. The method of claim 32, wherein the final layer is a lectin layer.

40. (canceled)

41. The method of claim 39, further comprising contacting the multilayer film with a ligand of the lectin and maintaining the multilayer film under conditions in which all or a portion of the lectin in one or more final layer or layers of the multilayer film is released, thereby exposing charged groups of an underlying polymer layer.

42. The method of claim 41, wherein:
a) the polymer is porcine gastric mucin, the lectin is wheat germ agglutinin and the ligand is N-Acetyl-D-Glucosamine; or
b) the polymer is bovine submaxillary mucin (BSM), the lectin is jacalin and the ligand is melibiose.

43-59. (canceled)

60. A method of detecting or isolating a molecule of interest, comprising:
a) attaching streptavidin to the molecule of interest;
b) contacting the molecule of interest with the multilayer film of claim 1, wherein the film further comprises biotin attached to one or more of the lectin layers, thereby producing a combination; and
c) maintaining the combination under conditions in which the biotin of the multilayer film binds to the streptavidin of the molecule of interest,
thereby detecting or isolating a molecule of interest.

61-64. (canceled)

65. A method of reducing bacterial adhesion to a surface, comprising applying the multilayer film of claim 1 to the surface and depleting the lectin from the film.

66. (canceled)

67. A method of binding a microorganism, comprising contacting the microorganism with the multilayer film of claim 1.

68-70. (canceled)
